(19) United States
(12) Patent Application Publication
(10) Pub. No.: US 2007/0218131 A1
(43) Pub. Date: Sep. 20, 2007

(54) EFFERVESCENT FORMULATIONS COMPRISING DESMOPRESSIN

(75) Inventor: Finn Larsen, EDINBURGH (GB)

Correspondence Address: NIXON PEABODY LLP - PATENT GROUP, CLINTON SQUARE, P.O. BOX 31051, ROCHESTER, NY, 14603-1051, US

(73) Assignee: ARDANA BIOSCIENCE LIMITED, EDINBURGH (GB)

(21) Appl. No.: 10/593,245

(22) PCT Filed: Mar. 18, 2005
(86) PCT No.: PCT/GB05/01136

§ 371 Date: Dec. 14, 2006

(30) Foreign Application Priority Data
Mar. 18, 2004 (GB) 0406048.9

(51) Int. Cl. A61K 9/46; A61K 38/12

(52) U.S. Cl. 424/466; 514/11

(57) ABSTRACT

An effervescent formulation comprising desmopressin, preferably comprising multilayer effervescent microspheres containing an acidic substance, a basic substance and water-soluble isolating agent. An effervescent formulation comprising desmopressin wherein dissolution in water of the multilayer effervescent microspheres leads, after almost immediate effervescence, to a solution or a homogeneous dispersion of the desmopressin. The formulation is used for treating diabetes insipidus, nocturnal enuresis, postoperative polyuria or polydipsia, nocturia associated with multiple sclerosis, mild to moderate haemophilia or von Willebrand's disease.

This invention relates to formulations of desmopressin and their use in the treatment of diabetes insipidus, nocturnal enuresis (night time bed wetting), postoperative polyuria or polydipsia, nocturia associated with multiple sclerosis, mild to moderate haemophilia and von Willebrand's disease. The formulations may also be used in tests of renal function and tests of fibrinolytic response.

The present treatment of diabetes insipidus with desmopressin typically requires three 100 μg doses to be given orally, and the treatment of nocturnal enuresis (usually in children) requires a dose of 200 μg to 400 μg when administered orally, or of 20 μg to 40 μg when administered intranasally.

Thus, the inventor considers it to be desirable for new formulations to be produced which have improved oral bioavailability and which allow for oral administration of lower doses and/or fewer doses, which will aid patient compliance.

The listing or discussion of a prior-published document in this specification should not necessarily be taken as an acknowledgement that the document is part of the state of the art or is common general knowledge

A first aspect of the invention provides an effervescent formulation comprising desmopressin. It may be used for the treatment of diabetes insipidus, nocturnal enuresis, postoperative polyuria or polydipsia, nocturia associated with multiple sclerosis, mild to moderate haemophilia and von Willebrand's disease. It may also be used in tests of renal function and tests of fibrinolytic response.

By ‘effervescent formulation’ we mean that the formulation is effervescent when placed in an aqueous solution.

By ‘desmopressin’ we include the compound 1-(3-mercaptopropanloic acid)-8-D-arginine vasopressin (also called 8-D-arginine-1-(3-mercaptopropanoic acid) vasopressin and 1-desamino-8-d-arginine vasopressin. It has the structure:

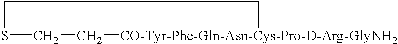

Also included are salts thereof, including acetate and citrate and other pharmaceutically acceptable salts.

Effervescent formulations offer an advantage over the existing forms of supplying desmopressin as they have a high level of patient acceptability. Effervescent formulations also give a more consistent pharmacokinetic profile than buccal delivery. The formulations may be placed on the tongue where they effervesce, and release the desmopressin.

A preferred embodiment of the invention is that the effervescent formulation comprises multilayer effervescent microspheres. The manufacture of certain suitable multilayer effervescent microspheres is described in WO 98/31342 and U.S. Pat. No. 6,210,711 B1, hereby incorporated by reference in their entirety.

A still further embodiment of the invention is that the multilayer effervescent microspheres contain an acidic substance, a basic substance and water-soluble isolating agent.

The term ‘microsphere’ will be intended to refer to microgranules formed of a support material consisting of a matrix in which the desmopressin is dispersed (eg within or outside the microsphere). In accordance with the European Pharmacopoeia monograph on spheres, microspheres have an average diameter of less than 1.0 mm and greater than or equal to 1.0 μm. They are generally intended for oral or parenteral administration and are used either as constituents of pharmaceutical form, such as tablets, or in their natural form combined or otherwise with other excipients, and distributed or otherwise in unit doses, such as sachets, gel-capsules or powder for injectable preparation.

The ‘water-soluble isolating agent’ may be any such agent which serves as both a binder and as an isolating barrier intended to avoid an effervescent reaction between the alkaline substance and the acidic substance during the preparation process but also during storage of the microspheres, irrespective of the storage conditions. Typically, it is chosen from polvinylpyrrolidone, hydroxypropyl cellulose, methyl cellulose, lactose and sucrose.

By ‘acidic substance’ we include a powder of acidic nature containing an organic acid, for example citric acid, ascorbic acid or acetylleucine.

By ‘basic substance’ we mean a powder of alkaline nature containing a sodium bicarbonate or any other carbonate usually used in the preparation of effervescent forms, such as lithium hydrogen carbonate, monosodium carbonate, lithium glycine carbonate, monopotassium carbonate, calcium carbonate or magnesium carbonate. It is preferred that the ‘basic substance’ is a sodium salt such as sodium bicarbonate.

A preferred embodiment of the invention relates to multilayer effervescent microspheres containing an acidic substance, a basic substance and a water-soluble isolating agent whose dissolution in water leads, after almost immediate effervescence, to a solution or a homogeneous dispersion of desmopressin.

According to a first variant of this embodiment of the invention, the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

According to a second variant of this embodiment of the invention, the water-soluble isolating agent is in the form of a thin film separating the acidic and alkaline substances. In this case, each microsphere has a three-layer structure: a layer of acidic substance and a layer of alkaline substance separated by a layer of water-soluble isolating agent.

Whether the microspheres have a two-layer or three-layer structure, the water-soluble isolating agent serves two purposes; it acts as a binder and as an isolating barrier intended to avoid an effervescence reaction between the alkaline substance and the acidic substance during the preparation process but also during storage of the microspheres, irrespective of the storage conditions.

In a preferred embodiment of the invention the effervescent formulation contains desmopressin present in a unit dose amount of from about 0.1 to 150 μg such as 0.1 μg, 0.2 μg, 0.3 μg, 0.4 μg, 0.5 μg, 0.6 μg, 0.7 μg, 0.8 μg, 0.9 μg, 1 μg, 1.5 μg, 2 μg, 3 μg, 5 μg, 7.5 μg, 10 μg, 20 μg, 30 μg, 40 μg, 50 μg, 60 μg, 70 μg, 80 μg, 90 μg, 100 μg, 110 μg, 120 μg, 130 μg, 140 μg or 150 μg. It will be appreciated that the unit dose (or daily dose) will vary depending on the use to which the effervescent formulation is to be put, for example the disease to be treated.

Thus, for the treatment of diabetes insipidus, the patient may be administered initially around 30 μg daily, whereas the maintenance dose may be from around 30 μg to 60 μg daily, with a range of from around 20 to 120 μg. It is preferred that a single dose of around 30 μg is used, although two intermittent doses of 15 μg or three intermittent doses of 10 μg may also be used. This dose is around ten fold lower than with existing oral treatment regimes. Typically the effervescent formulation is placed on the tongue.

For the treatment of nocturnal enuresis (particularly if urine concentrating ability is normal) the dose may be 20 μg at bedtime, increased to 40 μg if the lower dose is not effective. This dose is around ten fold lower with existing oral treatment regimes. Typically the effervescent formulation is placed on the tongue.

Typically, the dose administered using the effervescent formulation is similar to the dose administered using intranasal formulations, except that there is better patient compliance.

For the treatment of postoperative polyuria or polydipsia, the dose may be adjusted to urine osmolality.

For the treatment of nocturia associated with multiple sclerosis, typically 10 μg to 20 μg is administered at bedtime. For renal function testing, typically 40 μg are given to adults, 20 μg is given to a child between the age of 1 and 15 years, and 10 μg is given to an infant under one year. Preferably the effervescent formulation is placed on the tongue.

In a further embodiment the effervescent formulation of the invention is presented in a tablet form. Methods of forming tablets suitable for the invention from such an effervescent formulation are well known to those skilled in the art. Methods of forming tablets suitable for the invention from such an effervescent formulation are well known to those skilled in the art. A tablet may be made by compression or moulding, optionally with one or more accessory ingredients. Compressed tablets may be prepared by compressing in a suitable machine the active ingredient in a free-flowing form such as a powder or granules. Moulded tablets may be made by moulding in a suitable machine a mixture of the powdered compound moistened with an inert liquid diluent.

In a further embodiment the effervescent formulation of the invention is presented in a powder form. Methods of forming powders suitable for the invention from such an effervescent formulation are well known to those skilled in the art.

It is preferred that when the formulation contains microspheres, the desmopressin is not present within the microspheres. For example, when microspheres are tabletted to form a tablet, the desmopressin is preferably present on or between the microspheres in the tablet.

The desmopressin may, however, in some embodiments, be present in the microspheres.

A further aspect of the invention is a process for making an effervescent formulation containing desmopressin.

A preferred embodiment of the invention is a process wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of desmopressin.

In a preferred embodiment of the invention, the desmopressin is not present within the microspheres. In a further embodiment of the process of the invention the acidic and/or basic substances contains or contain desmopressin.

In a further preferred embodiment of the process of the invention the process employs the method of rotary granulation in a fluidized air bed.

The advantage of rotary granulation applied to these effervescent compositions is the continuous linking of the operations in one and the same chamber which, as a result of the components used and certain precautions taken, induces no effervescence. Furthermore, this rotary granulation technique allows the relative proportions of the various compounds to be modified, in particular the relative molar proportions of the acidic and basic fractions.

Specifically, the process according to the invention makes it possible advantageously to obtain effervescent forms whose relative proportion of alkaline and acidic fractions is less than the stoichiometric proportion implemented in the prior art for effervescent tablets manufactured by the granulation method, without the quality of the effervescence being adversely affected.

In particular, the relative proportion of the basic and acidic substances implemented in the context of the process according to the invention is less than 0.6, in particular less than 0.25.

All the steps of the process according to the invention are carried out under atmospheric pressure, without any specific dehydration system or any specific precautions.

The apparatus used to carry out the process for preparing the effervescent microspheres is, for example, apparatus constructed by the company Glatt, onto which a rotor tank is fitted.

Such an item of apparatus is described in patent EP 0,505,319, which we include, by way of reference, in the present application.

Also subject of the present invention is, firstly, a process for preparing effervescent microspheres which have a two-layer structure according to the first variant described above.

Said process is performed by rotary granulation in a fluidized air bed combined with a system for spraying powder and a system for the tangential spraying of wetting liquid. The process comprises two continuous steps, a first step of spheronization of microspheres using a powder A and a second step of spheronization of a powder B on the microspheres of powder A, one of the powders A and B being acidic and the other alkaline and it being possible for each of them to contain or consist of desmopressin. It is preferred that powders A or B contain but do not consist of desmopressin.

During the first spheronization, the powder A is placed in the moving rotary granulation tank and suspended in the air bed. The components of the powder A are mixed together for five minutes and the air inlet temperature is stabilised to a temperature T₀.

The powder A thus blended is sprayed with a wetting liquid containing the water-soluble isolating agent. The microspheres of powder A obtained are dried by bringing the air inlet temperature to Ts and are then optionally screened using a 1000 μm screen. During the second spheronization, the air inlet temperature is brought to T₀. The powder B and the wetting liquid containing the water-soluble isolating agent are then simultaneously sprayed onto the dried powder A microspheres obtained previously. The powder B is sprayed by means of the powder spraying system installed on the Glatt apparatus. The two-layer microspheres obtained are dried by bringing the air inlet temperature to Ts. After drying, the microspheres must be packaged quickly, but a small amount of moisture uptake does not harm the storage.

During the two spheronizations, the wetting liquid containing the water-soluble isolating agent is the same, for example polyvinylpyrrolidone (PVP) dissolved in an alcohol or an aqueous-alcoholic mixture, in particular PVP dissolved to 4% by weight in ethanol at 60% by volume.

The two-layer microspheres obtained according to the process of the invention have an average particle size of between 20 and 500 μm.

A subject of the present invention is also a process for preparing effervescent microspheres which have a three-layer structure according to the second variant described above.

Said process is performed according to the method of rotary granulation in a fluidized air bed combined with a system for the tangential spraying of wetting liquid.

The process comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline and it being possible for each of them to contain or consist of desmopressin. It is preferred that powders A or B contain but do not consist of desmopressin.

During the first spheronization, the powder A containing an added binder, for example PVP, is placed in the moving tank and suspended in the air bed. The components of the powder A are mixed together for five minutes and the air inlet temperature is stabilized to T₀. The powder A thus blended is sprayed with a wetting liquid. The microspheres of powder A obtained are dried by bringing the air inlet temperature to Ts. During the second spheronization, the air inlet temperature is brought to T₀. The water-soluble isolating agent is added directly to the tank and the wetting liquid sprayed until microspheres of powder A which are coated with a film of water-soluble isolating agent are obtained, and are dried by bringing the air inlet temperature to Ts. After drying, the coated microspheres are screened and the powder B is then added directly to the rotary granulation tank when the air inlet temperature has stabilized at T₀. The three-layer microspheres are obtained by spraying the preceding microspheres with a wetting liquid. The three-layer microspheres obtained are dried by bringing the air inlet temperature to Ts. After drying, the microspheres must be packaged quickly, but a small amount of moisture uptake does not harm the storage.

During the first two steps, the wetting liquid is, for example, an aqueous-alcoholic solution, in particular ethanol at 60% by volume. During the final step, the water-soluble isolating agent can be introduced by means of the powder B, in which case the wetting liquid used will be the same as during the first two steps, or alternatively the isolating agent is introduced by means of the wetting liquid, which will be an alcoholic or aqueous-alcoholic solution containing the isolating agent, for example PVP dissolved to 4% by weight in ethanol at 60% by volume.

The three-layer microspheres obtained according to the process of the invention have an average particle size of between 200 and 1000 μm.

According to the process for manufacturing microspheres, whether they are two-layer or three-layer microspheres, the powder of alkaline nature contains a sodium bicarbonate or any other carbonate usually used in the preparation of effervescent forms, such as lithium hydrogen carbonate, monosodium carbonate, lithium glycine carbonate, monopotassium carbonate, calcium carbonate, magnesium carbonate and, optionally desmopressin; whereas the powder of acidic nature contains an organic acid, for example citric acid, ascorbic acid, acetylleucine and, optionally, desmopressin. It is preferred that the desmopressin is not present within the microspheres, but rather is present on or between them in the final formulation (typically a tablet). In some embodiments, however, the powder of alkaline nature and the powder of acidic nature contain but do not consist of desmopressin.

In a further embodiment of the process of the invention the acidic and alkaline powders can also contain a diluent, for example lactose or Glucidex; flavorings and sweeteners, for example orange flavoring, citric acid, sodium saccharinate; various excipients.

In a preferred embodiment of the process of the invention desmopressin is present such that the resulting effervescent formulation contains desmopressin present in a unit dose amount of from about 0.1 to 150 μg such as 0.1 μg, 0.2 μg, 0.3 μg, 0.4 μg, 0.5 μg, 0.6 μg, 0.7 μg, 0.8 μg, 0.9 μg, 1 μg, 1.5 μg, 2 μg, 3 μg, 5 μg, 7.5 μg, 10 μg, 20 μg, 30 μg, 40 μg, 50 μg, 60 μg, 70 μg, 80 μg, 90 μg, 100 μg, 110 μg, 120 μg, 130 μg, 140 μg or 150 μg.

In a further embodiment of the process of the invention the effervescent formulation of the invention is presented in a tablet form. Methods of forming tablets suitable for the invention from such an effervescent formulation are well known to those skilled in the art as described above. Preferably, the desmopressin is present in the tablet on or between microspheres (when present).

According to one embodiment of the invention, the powder A is of alkaline nature and the powder B is of acidic nature.

According to another embodiment of the invention, the powder B is of alkaline nature and the powder A of acidic nature.

The wetting liquid is sprayed by means of a nozzle 1.2 mm in diameter, at an average flow rate of between 10 and 30 g/min. The air inlet temperature of the fluidized bed is between 55 and 65° C. during the spheronization steps (T₀) and between 75 and 85° C. during the drying phases (Ts).

The microspheres obtained according to the process of the invention contain 5 to 75% of alkaline substance, 10 to 75% of acidic substance, 3 to 15% of water-soluble isolating agent, 5 to 50% of diluent and 1 to 30% of flavorings and sweeteners and may, in some embodiments, contain an appropriate amount of desmopressin such as 0.02 to 0.1%, typically around 0.05%. Thus, a 100 mg tablet may comprise 80 mg of microspheres which may contain around 40 μg desmopressin.

The relative humidity of the microspheres obtained according to the process of the invention, measured for fifteen minutes by the infrared balance method at 90° C., is between 1 and 2% at the rotary granulation tank outlet.

The overall yield for the process is calculated from the fraction of particles smaller than 2500 μm in size, the working yield of the spheres corresponds to the fraction of particles between 200 and 1000 μm, for the process for preparing three-layer microspheres, between 20 and 500 μm for the process for preparing two-layer microspheres.

The feasibility of the process according to the invention is evaluated according to the ease with which the microspheres are obtained, the speed of production of a batch and the yield for each step.

Analysis of the batches includes particle size analysis of a sample of 100 g of spheres by the superimposed screens method (sample obtained from the total fraction of a batch), after which a morphological study of the microspheres obtained, relating to the overall appearance, sphericity, cohesion and uniformity of the particles, is carried out by examination with a binocular magnifying glass.

According to one variant of the invention, the two-layer or three-layer effervescent microspheres are manufactured by the mounting technique combined with a system for the tangential spraying of wetting liquid. The powder A and the powder B can be mounted successively on spheres containing desmopressin coated with water-soluble isolating agent, or on neutral spheres.

A further aspect of the invention is an effervescent formulation of desmopressin obtained or obtainable by any one of the processes of the invention mentioned above.

A further aspect of the invention provides an effervescent formulation of desmopressin for use in medicine.

A further aspect of the invention provides a pharmaceutical composition which comprises an effervescent formulation of desmopressin according to the invention and a pharmaceutically acceptable carrier.

A further aspect of the invention is a method of treating diabetes insipidus, nocturnal enuresis, postoperative polyuria or polydipsia, nocturia associated with multiple sclerosis, mild to moderate haemophilia or von Willebrand's disease by administering an effervescent formulation of desmopressin according to the invention and/or obtained or obtainable by a process according to the invention.

A further aspect of the invention is the use of an effervescent formulation of desmopressin according to the invention and/or obtained or obtainable by a process according to the invention in the manufacture of a medicament for treating diabetes insipidus, nocturnal enuresis, postoperative polyuria or polydipsia, nocturia associated with multiple sclerosis, mild to moderate haemophilia or von Willebrand's disease.

It is preferred if the formulation is used to treat diabetes insipidus, particularly pituitary (cranial) diabetes insipidus. It is also preferred if the formulation is used to treat nocturnal enuresis, particularly primary nocturnal enuresis.

Typically, for the treatment of diabetes insipidus, the patient takes a daily tablet which is placed on the tongue to deliver to the patient an effective amount of desmopressin for ameliorating the disease.

Typically, for the treatment of nocturnal enuresis, the patient takes a daily tablet (at bedtime) which is placed on the tongue to deliver to the patient an effective amount of desmopressin to control bed wetting.

Preferred embodiments of the invention are described in the following processes.

Process 1: Process for preparing multilayer effervescent microspheres containing an acidic substance, a basic substance, and a water-soluble isolating agent which upon dissolution in water leads, after almost immediate effervescence, to a solution or a homogeneous dispersion of desmopressin, wherein the acidic and basic substances contain or consist of desmopressin, which employs the method of rotary granulation in a fluidized air bed.

Process 2. Process for preparing microspheres defined in process 1, which employs the method of rotary granulation in a fluidized air bed combined with a system for spraying powder and a system for the tangential spraying of wetting liquid, which comprises two continuous steps, a first step of spheronization of microspheres using a powder A and a second step of spheronization of a powder B on the microspheres of powder A, one of the powders A and B being acidic and the other alkaline.

Process 3. Process according to process 2, wherein the powder A is introduced directly into the rotary granulation tank and then sprayed with a wetting liquid containing the water-soluble isolating agent, while the powder B and a wetting liquid containing the water-soluble isolating agent are simultaneously and respectively sprayed via the system for spraying powder and the system for the tangential spraying of liquid.

Process 4. Process according to process 3, wherein the microspheres obtained have an average particle size of between 20 and 500 μm.

Process 5. Process for preparing microspheres as defined in process 1, which employs the method of rotary granulation in a fluidized air bed combined with a system for the tangential spraying of wetting liquid, which comprises three continuous steps, a first step of spheronization of microspheres using a powder A, a second step of spheronization of a water-soluble isolating agent on the microspheres of powder A, and then a third step of spheronization of a powder B on the microspheres A protected with a film of water-soluble isolating agent, one of the powders A and B being acidic and the other alkaline.

Process 6. Process according to process 5, wherein the powder A and the water-soluble isolating agent are sprayed with an alcoholic or aqueous-alcoholic solution.

Process 7. Process according to process 5, wherein the powder B contains the water-soluble isolating agent and is sprayed with an alcoholic or aqueous-alcoholic solution.

Process 8. Process according to process 5, wherein the powder B is sprayed with a wetting liquid containing the water-soluble isolating agent.

Process 9. Process according to process 5, wherein the microspheres obtained have an average particle size of between 200 and 1000 μm.

Process 10. Process according to process 3, wherein the wetting liquid containing the water-soluble isolating agent is polyvinylpyrrolidone dissolved in an alcohol or an aqueous-alcoholic mixture, which is polyvinylpyrrolidone dissolved to 4% by weight in ethanol at 60% by volume.

Process 11. Process according to process 2 or 5, wherein the powder of alkaline nature contains a sodium bicarbonate or another carbonate used in the preparation of effervescent forms, selected from lithium hydrogen carbonate, monosodium carbonate, lithium glycine carbonate, monopotassium carbonate, calcium carbonate, magnesium carbonate; and desmopressin.

Process 12. Process according to process 2 or 5, wherein the powder of acidic nature contains citric acid or ascorbic acid or, acetylleucine, and/or desmopressin.

Process 13. Process according to process 1, wherein the powder of alkaline nature also contain an edible diluent and; flavorings and sweeteners.

Process 14. Process according to process 2 or 5, wherein the microspheres obtained contain 5 to 75% of alkaline substance, 10 to 75% of acidic substance, 3 to 15% of water-soluble isolating agent, 5 to 50% of diluent, and 1 to 30% of flavorings and sweeteners.

Process 15. Process according to process 2 or 5, wherein the powder A is of alkaline nature and the powder B of acidic nature.

Process 16. Process according to process 2 or 5, wherein the powder A is of acidic nature and the powder B of alkaline nature.

Process 17. Process according to process 3 or 6, wherein the wetting liquid is sprayed by means of a nozzle 1.2 mm in diameter, at an average flow rate of between 10 and 30 g/min.

Process 18. Process according to process 2 or 5, wherein the air inlet temperature of the fluidized bed is between 55 and 65° C. during spheronization steps, and between 75 and 85° C. during drying phases associated with the spheronization steps.

Process 19. Process according to process 2 or 5, wherein the relative humidity of the microspheres obtained is between 1 and 2% at the rotary granulation tank outlet.

Process 20. Process for preparing microspheres as defined in process 1, which employs the mounting technique combined with a system for the tangential spraying of wetting liquid.

Process 21. Process according to process 20, wherein the powder A and the powder B are mounted successively on spheres containing desmopressin coated with water-soluble isolating agent, or on neutral spheres.

Process 22. Process according to process 12, wherein the powder of acidic nature also contains an edible diluent and flavorings and sweeteners.

Microspheres can also be made using the above processes where desmopressin is not present (ie a basic and acidic substance which does not contain desmopressin is used).

The examples which follow illustrate the invention without limiting its scope.

The percentages are expressed on a weight basis.

### EXAMPLE 1

Two-layer effervescent microspheres containing ascorbic acid (vitamin C)

Alkaline microspheres are prepared, on which is deposited the acidic substance (vitamin C).

The table below gives the details of the formulation used.

| FORMULATION | COMPONENT | PERCENTAGE |
|---|---|---|
| Powder A | | |
| Alkaline compound | Sodium bicarbonate | 20% |
| Diluent | Lactose | 6% |
| Sweetener | Glucidex 6 .RTM. | 6% |
| Powder B | | |
| Acidic compound | Ascorbic acid | 50% |
| Flavoring | Orange flavoring | 1% |
| Sweeteners | Sodium saccharinate | 0.3% |
| | Glucidex 6 .RTM. | 6.35% |
| Diluent | Lactose | 6.35% |

The wetting liquid used during the two successive rotary granulations is an aqueous-alcoholic PVP solution containing 4% PVP in ethanol at 60% by volume.

This mixture is sprayed at an average flow rate of 25 grains per minute.

In this formulation, the lactose is combined in equal part with Glucidex 60, although it is possible to use lactose alone.

The powder formulations A and B are prepared on batches of variable size of 1000 to 5000 g with, depending on the case, use of equipment from the company Glatt.

The effervescent spheres obtained have a fairly uniform appearance and a majority particle size of fractions between 200 and 500 μm. The relative humidity is 1.6% at the rotary granulation tank outlet.

### EXAMPLE 2

Two-layer effervescent microspheres containing acetylleucine

Alkaline microspheres are prepared, on which is deposited the acidic substance (acetylleucine) under the same conditions as in Example 1.

The table below gives the details of the formulation used.

| FORMULATION | COMPONENT | PERCENTAGE |
|---|---|---|
| Powder A | | |
| Alkaline compound | Sodium bicarbonate | 20% |
| Diluent | Lactose | 9.85% |
| Powder B | | |
| Acidic compound | Acetylleucine | 50% |
| Flavoring | Orange flavoring | 1% |
| Sweetener | Sodium saccharinate | 0.3% |
| Diluent | Lactose | 9.85% |

The particle size distribution of the batch is a majority for the fractions 25 to 500 μm.

The relative humidity is 1.9% at the rotary granulation tank outlet.

According to the size of the batches ranging from 1000 to 10,000 g, apparatus GPCG 1 or GPCG 5 from the company Glatt with a rotor tank mounting [lacuna].

### EXAMPLE 3

Three-layer effervescent microspheres containing ascorbic acid (vitamin C)

Three-layer effervescent microspheres are manufactured, comprising an alkaline core isolated from the acidic substance, ascorbic acid, by means of a film of PVP.

| FORMULATION | COMPONENT | PERCENTAGE |
|---|---|---|
| Powder A | | |
| Alkaline compound | Sodium bicarbonate | 25% |
| Binder | PVP K30 | 1.316% |
| Diluent | Lactose | 7.950% |
| Water-soluble | PVP K30 | 6.958% |
| isolating agent | | |
| Powder B | | |
| Acidic compound | Ascorbic acid | 50% |
| Flavoring | Orange flavoring | 1% |
| Sweeteners | Sodium saccharinate | 0.2% |
| | Citric acid | 1% |
| Diluent | Lactose | 6.950% |

The test is carried out in apparatus of GPCG1 type from the company Glatt, with the rotor tank mounting.

1460 g of ethanol at 60% by volume are sprayed in total during the three steps, at an average flow rate of 15 grams per minute.

The size of the final batch is 1000 g.

The working yield corresponding to the fraction of particles between 200 and 1000 μm is 65%. The relative humidity is 1.5% at the tank outlet.

### EXAMPLE 4

Three-layer effervescent microspheres containing citric acid.

Three-layer effervescent microspheres are manufactured, comprising an alkaline core (basic effervescent agent) isolated from the acidic substance (acidic effervescent agent), citric acid, by means of a film of PVP (binder). Flavouring, sweetening substance and lubricant is present when the microspheres are prepared into a tablet in the amounts given.

| Property | Component | Amount (%) |
|---|---|---|
| Acid effervescent agent | Citric acid | 62.31 |
| Basic effervescent agent | Sodium bicarbonate | 25.85 |
| Binder | Polyvinylpyrrolidone | 5.34 |
| | K30 Kollidon 30 | |
| Flavour | Mint EH0159 E40159 | 3.00 |
| Sweetening substance | Aspartame | 1.50 |
| Lubricant | Magnesium stearate | 1.00 |
| Lubricant | Talc 00 | 1.00 |
| | TOTAL | 100 |

### EXAMPLE 5

### Preparation of Effervescent Tablets Containing Desmopressin

Effervescent tablets containing between 10 μg and 30 μg desmopressin are prepared so that the time for drug dissolution or tablet disintegration and/or dissolution is less than 10 minutes.

In the laboratory scale manufacturing process, desmopressin is mixed with effervescent microspheres prepared as described above (eg using the proportion of acid substance, basic substance and isolating substance as described in Example 4) with a Glatt GPCGI. The mixture is then added with diluent (mannitol), lubricants (magnesium stearate, talc), flavouring and tabletted on a single punch alternative press under isolator.

In the industrial scale process, desmopressin is introduced directly on the effervescent microspheres directly in the Glatt by the powder device. After drying, the spheres are mixed with the other excipients and tabletted.

### EXAMPLE 6

### Treatment of Nocturnal Enuresis with Desmopressin

Effervescent tablets are made containing 20 μg using the process described in Example 5. They are administered at bed time by placing on the tongue of the patient.

### EXAMPLE 7

### Treatment of Diabetes Insipidus with Desmopressin

Effervescent tablets are made containing 10 μg desmopressin using the process described in Example 4. They are administered once or twice a day is by placing on the tongue of the patient.

1-28. (canceled)

29. An effervescent formulation comprising desmopressin and multilayer effervescent microspheres.

30. An effervescent formulation according to claim 29 wherein the multilayer effervescent microspheres contain an acidic substance, a basic substance, and a water-soluble isolating agent.

31. An effervescent formulation according to claim 30 wherein dissolution in water of the multilayer effervescent microspheres leads, after almost immediate effervescence, to a solution or a homogeneous dispersion of the desmopressin.

32. An effervescent formulation according to claim 31 wherein the water-soluble isolating agent is dispersed in the entire bulk of each microsphere, the latter having a two-layer structure: a layer of acidic substance in which is dispersed the water-soluble isolating agent and a layer of alkaline substance in which is dispersed the water-soluble isolating agent.

33. An effervescent formulation according to claim 31 wherein the water-soluble isolating agent is in the form of a thin film separating the acidic and alkaline substances such that each microsphere has a three-layer structure: a layer of acidic substance and a layer of alkaline substance separated by a layer of water-soluble isolating agent.

34. An effervescent formulation according to claim 29 wherein the desmopressin is present in a unit dose amount of from 1 μg to 1500 μg.

35. An effervescent formulation according to claim 34 wherein the desmopressin is present in a unit dose amount of 100 μg to 400 μg.

36. An effervescent formulation according to claim 29 wherein the formulation is presented in a tablet form.

37. An effervescent formulation according to claim 29 wherein the formulation is presented in a powder form.

38. An effervescent formulation according to claim 29 wherein the desmopressin is present within a microsphere.

39. An effervescent formulation according to claim 29 wherein the desmopressin is not present within a microsphere.

40. A pharmaceutical composition comprising an effervescent formulation according to claim 29 and a pharmaceutically acceptable carrier.

41. A process for making an effervescent formulation containing desmopressin wherein the effervescent formulation comprises multilayer effervescent microspheres containing an acidic substance, a basic substance, and a water-soluble isolating agent which upon dissolution in water leads, after almost immediate effervescence, to a solution or a homogeneous dispersion of desmopressin.

42. A process according to claim 41 wherein the acidic and/or basic substances contains or contain desmopressin.

43. A process according to claim 41 wherein the desmopressin is not present in microspheres.

44. A process according to claim 42 which employs the method of rotary granulation in a fluidized air bed.

45. A process according to claim 41 wherein basic substance also contains an edible diluant and/or flavourings and/or sweeteners.

46. A process according to claim 41 wherein the desmopressin is present in an amount to give from 1 μg to 1500 μg in the final unit dosage form.

47. A process according to claim 46 wherein the desmopressin is present in an amount to give from 100 μg to 400 μg in the final unit dosage form.

48. A process according to claim 41 further comprising preparing the microspheres into a tablet.

49. A process according to claim 48 wherein the desmopressin is present on or between the microspheres in the tablet.

50. An effervescent formulation of desmopressin obtained by the process of claim 41.

51. A method of treating a condition selected from the group of diabetes insipidus, nocturnal enuresis, postoperative polyuria or polydipsia, nocturia associated with multiple sclerosis, mild to moderate haemophilia and von Willebrand's disease, said method comprising:
administering an effervescent formulation of claim 29 to a patient under conditions effective to treat the condition.
